(19) United States
(12) Patent Application Publication
(10) Pub. No.: US 2026/0354897 A1
(43) Pub. Date: Nov. 12, 2026

(54) SURGICAL DEVICE USING HIGH-FREQUENCY ELECTROMAGNETIC WAVES

(71) Applicant: SKINGRAB CO., LTD., Seoul (KR)

(72) Inventor: Sugun LEE, Paju-si (KR)

(73) Assignee: SKINGRAB CO., LTD., Seoul (KR)

(21) Appl. No.: 19/264,187

(22) Filed: Jul. 9, 2025

(30) Foreign Application Priority Data
May 7, 2025 (KR) 10-2025-0058833

(51) Int. Cl. A61B 18/18; A61B 18/00

(52) U.S. Cl. CPC A61B 18/18; A61B 2018/00029; A61B 2018/00047; A61B 2018/00476

(57) ABSTRACT

Disclosed is a non-invasive surgical device utilizing electromagnetic waves of high-frequency energy. The surgical device capable of delivering high-frequency energy during surface electrode-based radio frequency (RF) treatment includes an electrode unit that touches a surface of a skin, an energy supply unit that supplies energy to the electrode unit during a reference period, a cooling unit, and a processor that controls the energy supply unit and the cooling unit. The processor controls the energy supply unit such that an electromagnetic wave of a frequency of 2.40 to 2.50 GHz is generated by the electrode unit.

### CROSS-REFERENCE TO RELATED APPLICATIONS

A claim for priority under 35 U.S.C. § 119 is made to Korean Patent Application No. 10-2025-0058833 filed on May 7, 2025 in the Korean Intellectual Property Office, the entire contents of which are hereby incorporated by reference.

### BACKGROUND

Embodiments of the present disclosure described herein relate to a non-invasive surgical device utilizing electromagnetic waves of high frequency energy.

The content described below merely provides background information related to an embodiment of the present disclosure and does not constitute prior art.

Skin care devices for removing wrinkles, restoring skin elasticity, and removing sebum use a method (HIFU type) of delivering ultrasound to skin tissues, a method (RF type) of delivering high-frequency waves to skin tissues, and a method (Optical type) of irradiating laser light to skin tissues.

Conventional invasive devices that deliver high-frequency waves to skin tissues may repeatedly invade a deep portion (e.g., a dermal layer) of a skin with an RF needle electrode moving back and forth in a vertical direction, may remove damaged collagen, elastic fibers, or the like from the deep portion of the skin by using the heat generated by high-frequency waves, and may promote new formation. Furthermore, such skin care devices may help improve skin pigmentation, acne scars, and wrinkles.

However, conventional surgical devices have difficulty in concentrating energy on a specific skin depth or tissue due to dispersion of RF energy. In addition, most commercialized RF devices utilize relatively low-frequency bands (several MHz to several tens of MHz), and thus have limitations in penetration depth adjustment and energy efficiency.

For example, BTL's Vanquish focuses on abdominal fat reduction in a non-contact method using RF of 27.12 MHz, but may have limitations in treating specific areas due to low precision. DEKA's ONDA Coolwaves™ uses 2.45 GHz microwaves, but the effects may vary depending on skin type. Accordingly, it may be difficult to provide consistent results to all users. MiraDry is specialized in treating axillary hyperhidrosis by using 5.8 GHz microwaves, but it is difficult to apply to other areas or skin diseases.

The present disclosure provides the following technical differences compared to conventional technologies. First, the present disclosure adopts a coaxial antenna structure and a variable impedance matching circuit, and thus a precise energy transfer is possible compared with the conventional devices. Second, the present disclosure integrates a multi-step pulse control algorithm and a real-time temperature monitoring system to minimize skin damage while maximizing the treatment effect. Third, the present disclosure provides a precise epidermal protection function that the conventional devices such as ONDA Coolwaves™ and MiraDry may not provide, by introducing a hybrid cooling system that combines a Peltier element and a refrigerant injection. Fourth, the present disclosure may simultaneously target various skin depths through a dual frequency system of 2.45 GHz and 6.78 MHz, and thus may be applied to various skin diseases and cosmetic purposes compared with a conventional single frequency system. Accordingly, there is a need for an ultrasound providing device that supplies energy by targeting a specific depth of a skin.

There is a prior art disclosed as Korean Registered Patent No. 10-0985822 (2010 Oct. 8.).

### SUMMARY

Embodiments of the present disclosure provide a surgical device for skin treatment that is capable of delivering energy by targeting a specific depth of a skin.

Embodiments of the present disclosure provide a non-invasive skin surgical device that overcomes the limitations of conventional technologies, by precisely targeting a specific depth of the skin by using high-frequency electromagnetic waves of 2.40 to 2.50 GHz and maximizing energy transfer efficiency.

Embodiments of the present disclosure provide a surgical device for skin treatment that is capable of being applied to various skin types and conditions and provides safe and consistent treatment results.

Problems to be solved by the present disclosure are not limited to the problems mentioned above, and other problems not mentioned will be apparent by those skilled in the art from the following description.

According to an embodiment, a surgical device capable of delivering high-frequency energy during surface electrode-based radio frequency (RF) treatment includes an electrode unit that touches a surface of a skin, an energy supply unit that supplies energy to the electrode unit during a reference period, a cooling unit, and a processor that controls the energy supply unit and the cooling unit. The processor controls the energy supply unit such that an electromagnetic wave of a frequency of 2.40 to 2.50 GHz is generated by the electrode unit.

The energy supply unit includes an RF energy generation system including an oscillator and amplifier, and is designed to operate stably at 2.40 to 2.50 GHz.

In an embodiment, the processor may allow the cooling unit to cool the skin during a predetermined time after an electromagnetic wave is generated by the electrode unit.

In an embodiment, the processor may split the reference period into a first section and a second section based on a specific point in time within the reference period, and may control the cooling unit and the energy supply unit such that a cooling time in the first section is different from a cooling time in the second section.

The electrode unit may further include a dielectric material, such as glycerin gel, to improve contact with the skin and to increase energy transfer efficiency.

In an embodiment, the processor may allow the cooling unit and the energy supply unit such that the cooling time in the first section is longer than the cooling time in the second section.

In an embodiment, the cooling unit may adopt a contact cooling method using a Peltier element, and the processor may monitor a skin temperature and/or impedance in real time through a closed-loop control system, and thus may adjust the output of the energy supply unit and/or the cooling intensity of the cooling unit.

In an embodiment, the cooling unit may protect the skin by spraying an ultra-low temperature refrigerant onto the skin, and the processor may monitor a skin temperature and/or impedance in real time through a closed-loop control system, and thus may adjust the output of the energy supply unit and/or the cooling intensity of the cooling unit.

In an embodiment, the processor may control the energy supply unit such that the electromagnetic wave generation time in the second section is longer than the electromagnetic wave generation time in the first section.

In an embodiment, the surgical device may further include a temperature sensor that measures a temperature of a procedure area of the skin. The processor may switch to a third section when a temperature of the skin in the second section reaches a target temperature, and may supply low energy compared to the second section such that a temperature of the skin is uniformly kept without a cooling time in the third section.

In an embodiment, the processor may switch to a fourth section when a preset time elapses after switching to the third section, or there is a user request, and may control the energy supply unit and the cooling unit such that a temperature of the skin gradually decreases in the fourth section.

In an embodiment, the processor may allow the energy supply unit to generate at least one of an electromagnetic wave having frequencies of 2.40 to 2.50 GHz and an electromagnetic wave having frequencies of 6.70 to 6.80 MHz in the electrode unit.

In an embodiment, the processor may allow the energy supply unit to generate electromagnetic waves having the frequencies of 6.70 to 6.80 MHz in the first section, and may allow the energy supply unit to generate electromagnetic waves having the frequencies of 2.40 to 2.50 GHz in the second section.

In an embodiment, the processor may allow the energy supply unit to alternately generate electromagnetic waves having the frequencies of 2.40 to 2.50 GHz and electromagnetic waves having the frequencies of 6.70 to 6.80 MHz in the third section.

Besides, a computer program stored in a computer-readable recording medium for executing a method to implement the present disclosure may be further provided.

In addition, a computer-readable recording medium for recording a computer program for performing the method for implementing the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a drawing for describing a surgical device capable of delivering high-frequency energy during surface electrode-based RF treatment, according to an embodiment of the present disclosure;

FIG. 2 is a drawing illustrating a configuration of a surgical device capable of delivering high-frequency energy during surface electrode-based RF treatment, according to an embodiment of the present disclosure;

FIG. 3 is a schematic diagram showing an RF energy generation system and a structure of an antenna electrode unit of a surgical device, according to an embodiment of the present disclosure;

FIG. 4 is a flowchart showing a procedure method utilizing a plurality of frequencies in surface electrode-based RF treatment, according to an embodiment of the present disclosure;

FIGS. 5 to 7 are graphs showing treatment effects during surface electrode-based RF treatment, according to an embodiment of the present disclosure;

FIG. 8 is a graph illustrating hair reduction effects at 6 months after the last procedure;

FIG. 9 is a graph for comparing hair removal effects for each hair type;

FIG. 10 is a table for analyzing side effects after a procedure;

FIG. 11 is a conceptual diagram showing a change in penetration depth according to conductor length adjustment;

FIG. 12 is a graph showing a change in penetration depth according to cooling intensity;

FIG. 13 is a graph showing energy concentration according to an electrode phase difference; and

FIG. 14 is a graph showing a comprehensive effect of an RF energy penetration depth control mechanism.

### DETAILED DESCRIPTION

The same reference numerals denote the same elements throughout the present disclosure. The present disclosure does not describe all elements of embodiments. Well-known content in a technical field, to which the present disclosure belongs, or redundant content in which embodiments are the same as one another will be omitted. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components.

Throughout this specification, when it is supposed that a portion is “connected” to another portion, this includes not only a direct connection, but also an indirect connection. The indirect connection includes being connected through a wireless communication network.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above.

Unless there are obvious exceptions in the context, a singular form includes a plural form.

In each step, an identification code is used for convenience of description. The identification code does not describe the order of each step. Unless the context clearly states a specific order, each step may be performed differently from the specified order.

Hereinafter, operating principles and embodiments of the present disclosure will be described with reference to the accompanying drawings.

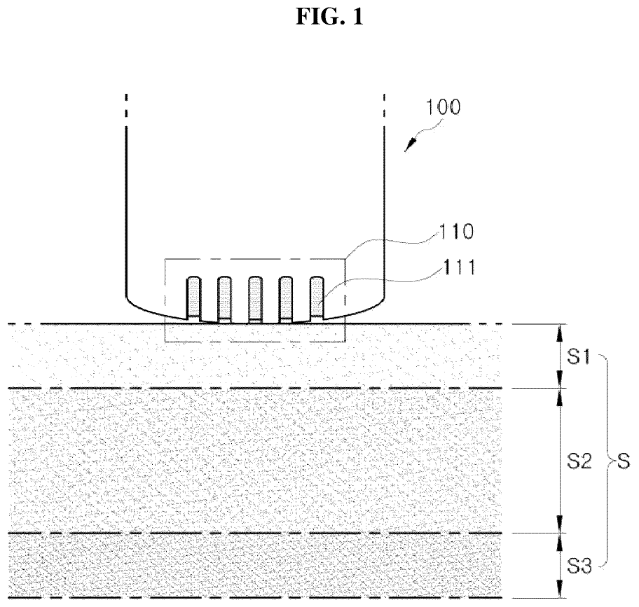

FIG. 1 is a drawing for describing an application environment of a surgical device 100 (hereinafter referred to as a ‘surgical device’) that may irradiate energy by targeting each skin depth during surface electrode-based RF treatment, according to an embodiment of the present disclosure.

The surgical device 100 may output high-frequency waves toward the inside of the skin for therapeutic or cosmetic purposes on a non-invasive manner. Here, the high-frequency waves may have a certain frequency band based on radio frequency (RF).

The surgical device 100 may output high frequencies of 2.40 to 6.80 GHz, but an embodiment is not limited thereto. However, in 2.45 GHz, the absorption rate of water molecules is high, and thus energy absorption occurs intensively in a fat layer. Accordingly, in an embodiment of the present disclosure, 2.45 GHz is adopted as a frequency.

In an embodiment, the surgical device 100 may irradiate RF energy in a range of 2.40 to 2.50 GHz. High frequencies of 2.40 to 2.50 GHz have the high absorption rate of water molecules, and thus energy absorption occurs intensively in the fat layer. In this way, the present disclosure may improve the fat reduction and cellulite treatment effects by focusing energy on skin tissue where fat is concentrated.

Because high-frequency waves of 2.40 to 2.50 GHz have very short wavelengths, it is easy to adjust the penetration depth, and thus the superficial layer (upper dermis) and the deep layer (subcutaneous fat layer) of the skin may be treated separately.

In an embodiment, high frequencies of 2.40 to 2.50 GHz may increase energy transfer efficiency at higher frequencies than conventional frequencies (e.g., 6.78 MHz).

In an embodiment, the high-frequency characteristics of 2.40 to 2.50 GHz may be utilized to achieve hyperhidrosis treatment or detailed skin texture improvement effects, which is difficult with conventional frequencies.

High frequencies of 2.40 to 2.50 GHz (especially 2.45 GHz) are greater than conventional RF frequencies (e.g., 6.78 MHz, 27.12 MHz) and provide higher energy transfer efficiency, shorter wavelengths, and easier penetration depth control. This makes it possible to treat a superficial layer (upper dermis) and a deep layer (subcutaneous fat layer) of the skin separately, and achieve various effects such as promoting collagen regeneration, destroying fat cells, and improving cellulite.

As may be seen, the surgical device according to an embodiment of the present disclosure may generate electromagnetic waves of a higher frequency than the frequency of electromagnetic waves generated by conventional surgical devices to promote various therapeutic effects.

The surgical device 100 may irradiate high-frequency pulsed shots to a skin ‘S’ by supplying power to an electrode unit 110 including a plurality of (surface) electrodes disposed on a location that is in contact with a skin surface. Here, a gap between the skin surface and the electrode (e.g., 111) may be generated by the structure (e.g., including housing) of the surgical device 100.

The surgical device 100 may reach energy not only to epidermis S1 of the skin ‘S’ but also to dermis S2 of the skin ‘S’, may remove damaged collagen, elastic fibers, or the like from the deep portion of the skin by using the heat generated by high-frequency waves and may promote new formation.

In the case of conventional surgical devices, it was difficult for the energy to reach subcutaneous fat S3 of the skin ‘S’. However, the surgical device 100 according to an embodiment of the present disclosure irradiates high frequency of 2.40 to 2.50 GHz, which has a high water molecule absorption rate, and thus energy may sufficiently reach the subcutaneous fat S3 of the skin ‘S’.

The surgical device 100 according to an embodiment of the present disclosure may supply power to the electrode unit 110 by dividing a reference period into a plurality of sections and relatively weakening the energy intensity for each section, without supplying strong power to the electrode unit 110 once during a reference period of treatment. Accordingly, skin damage may be reduced, skin temperature may be prevented from rising rapidly, and skin pain may be alleviated.

Here, the reference period may be set from 1 nanosecond to 1 minute, but an embodiment is not limited thereto, and the energy output per pulse shot may be from 0.1 watts to 500 watts, but an embodiment is not limited thereto.

As described above, the surgical device 100 may reduce skin damage by dividing the reference period into several sections to supply energy, rather than supplying strong energy to the skin at one time. Moreover, rapid increases in skin temperature may be prevented by controlling the energy intensity for each section, thereby alleviating skin pain.

In this case, even when multiple pulse shots are irradiated to the skin within the reference period of one-time treatment, the surgical device 100 according to an embodiment of the present disclosure may supply the total output energy amount used in the reference period of one-time treatment similarly to the case where power of strong intensity is supplied to the electrode unit 110 once within the reference period. Accordingly, rapid temperature rise of the skin may be prevented while excellent skin treatment effects are maintained.

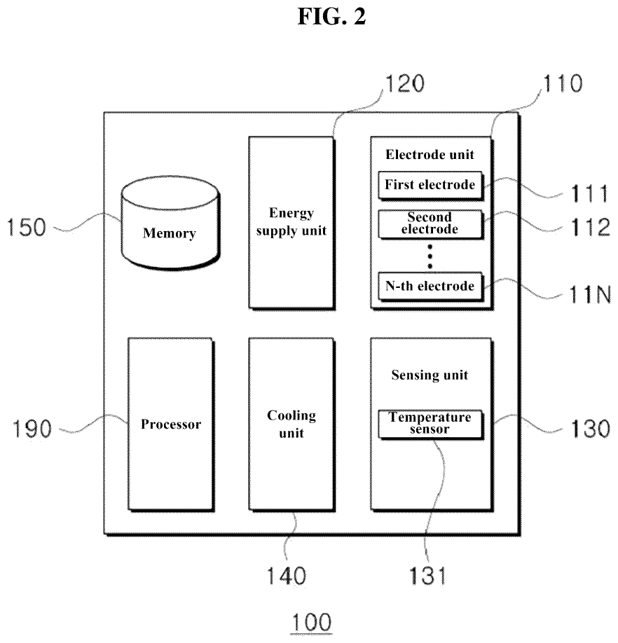

Hereinafter, the surgical device 100 will be described in more detail with reference to FIG. 2.

FIG. 2 is a block diagram showing a configuration of the surgical device 100, according to an embodiment of the present disclosure.

The surgical device 100 may include the electrode unit 110 including a plurality of electrodes 111 to 11N, an energy supply unit 120 for supplying power to the electrode unit 110, a sensing unit 130, a cooling unit 140, a memory 150, and at least one processor 190. The components of the surgical device 100 shown in FIG. 2 are not essential in implementing the surgical device 100. The surgical device 100 described herein may have more or fewer components than those listed above.

Because the electrode unit 110 including the plurality of electrodes 111 to 11N among the components is located on the surface of a skin, it may be referred to as a “surface electrode”, and may be placed inside the structure (e.g., housing) of the surgical device 100.

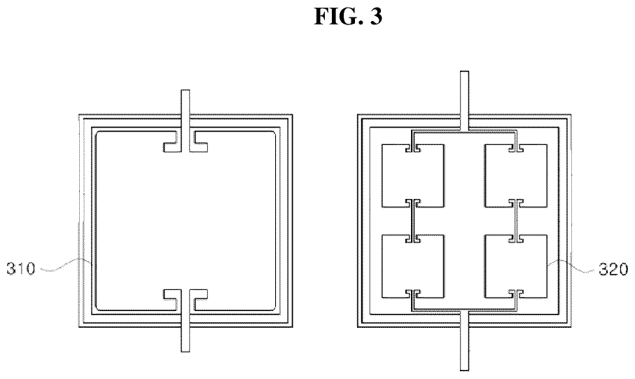

In an embodiment, referring to FIG. 3, the electrode unit may include one or more electrodes, and each electrode may be implemented in the form of a dipole antenna 310 or 320. The dipole antenna efficiently delivers RF energy of 2.45 GHz to a skin and is easy to manufacture due to its relatively simple structure. The arrangement of an electrode (antenna) may be implemented in various shapes such as circular, polygonal (e.g., triangular, square) or linear array, and may be optimized according to the treatment area and purposes. However, the present disclosure is not limited thereto.

The energy supply unit 120 includes an RF energy generation system. The RF energy generation system may consist of an oscillator and an amplifier. The oscillator generates a high-frequency signal in a range of 2.40 to 2.50 GHz (preferably 2.45 GHz), and the amplifier amplifies the signal generated by the oscillator and supplies the amplified signal to the electrode unit 110. The RF energy generation system needs to be designed to maintain stable frequency and output intensity. To this end, precise control circuits and feedback mechanisms may be applied. In an embodiment, the energy supply unit 120 may simultaneously supply energy to the plurality of electrodes 111 to 11N.

In another embodiment, the energy supply unit 120 may supply energy to a specific electrode. When the temperature of a skin area touching the specific electrode is greater than or equal to a predetermined value, the energy supply unit 120 may supply energy to another electrode so as to avoid the corresponding skin area and then may adjust a skin temperature increase. Moreover, the energy supply unit 120 may also supply energy to electrodes sequentially (or alternately) according to the specified order of electrodes.

The sensing unit 130 may be a module that detects environmental information about external environments and may representatively include a temperature sensor 131. When a skin procedure is performed by the surgical device 100, the temperature sensor 131 may detect the temperature of a procedure area.

In an embodiment, while monitoring the entire skin area of the procedure area, the temperature sensor 131 may sense an area or a point where the temperature exceeds a predetermined threshold value, but the present disclosure is not limited thereto.

The cooling unit 140 may be a module for lowering the skin temperature of all or part of the area of the procedure portion, and may adopt a contact cooling method using a Peltier element. The temperature of the skin surface may be lowered based on a cooling gas, but an embodiment is not limited thereto.

The Peltier element is a thermoelectric element that cools one side and heats the other side depending on the direction of current, and provides a cooling effect by absorbing heat through direct contact with the skin. The Peltier element is small, quiet, and easy to control temperature. The cooling unit 140 may increase cooling efficiency by including a heat sink and fan together with the Peltier element. In an embodiment, the cooling unit 140 may lower the temperature of the skin by contact cooling, such as a cooling plate or a cooling tip.

In an embodiment, the cooling unit 140 may lower the temperature of the skin by spraying a cryogenic refrigerant onto the skin.

In an embodiment, the cooling unit 140 may reduce the temperature of the skin by continuously circulating cold air over the surface of the skin.

The cooling unit 140 may lower the temperature of the skin by using at least one of the cooling methods described above, but the cooling method of the cooling unit 140 is not limited to the methods described above.

The memory 150 may store data supporting various functions of the surgical device 100 and a program for the operation of the processor 190, may store pieces of input/output data, and may store a plurality of application programs (or applications) running on the surgical device 100, data for operations of the surgical device 100, and commands for operations of the surgical device 100. The memory 150 may store one or more instructions and may include various deep learning-based models.

The memory 150 may include the type of a storage medium of at least one of a flash memory type, hard disk type, a solid state disk (SSD) type, a silicon disk drive (SDD) type, a multimedia card micro type, a memory of a card type (e.g., SD memory, XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disc. Furthermore, the memory 150 may be separate from the surgical device 100, but may be a database connected by wire or wirelessly, and may be implemented as a database system.

The processor 190 may include at least one core and may execute instructions stored in the memory 150. The processor 190 may be implemented with a memory that stores data regarding an algorithm for controlling operations of components within the surgical device 100, or a program for implementing the algorithm, and the at least one processor 190 that perform the above-described operation by using the data stored in the memory. In this case, each of the memory 150 and the processor 190 may be implemented as separate chips. Additionally, the processor 190 is a main controller that controls the overall operation of the surgical device 100, manages the user interface (UI), and performs data processing and storage.

At least one component may be added or deleted to correspond to the performance of the components illustrated in FIG. 2. Furthermore, it will be easily understood by those skilled in the art that mutual locations of the components may be changed to correspond to the performance or structure of the system.

In the meantime, each component shown in FIG. 2 refers to software components and/or hardware components such as field programmable gate array (FPGA) and application specific integrated circuit (ASIC).

From a clinical point of view, the above-described surgical device 100 may present a new method in which energy absorption occurs intensively in a fat layer. Accordingly, the surgical device 100 according to an embodiment of the present disclosure may achieve not only fat reduction and cellulite treatment effects, but also hyperhidrosis treatment and skin texture improvement effects.

In addition, the surgical device 100 is easy to control the penetration depth because it uses electromagnetic waves with a shorter wavelength than conventional surgical devices from a technical point of view. For this reason, the surgical device 100 may separately treat superficial and deep layers of the skin.

In an embodiment, the surgical device according to an embodiment of the present disclosure may include a variable impedance matching circuit capable of adjusting the distance between a central conductor and an outer conductor by adopting a coaxial antenna structure. The surgical device according to an embodiment of the present disclosure may include a coaxial antenna, a tunable waveguide, and an impedance matching circuit.

The coaxial antenna may include a coaxial structure consisting of a center conductor, a dielectric layer, and an outer conductor.

The tunable waveguide may implement a telescopic mechanism capable of adjusting the length of the central conductor from 0.5 mm to 5 mm.

The impedance matching circuit may control the penetration depth of RF energy into tissue by automatically adjusting antenna impedance.

When the conductor length is short (0.5 to 1.5 mm), the RF energy is concentrated on epidermis and upper dermal layers, thereby making it suitable for improving skin texture or treating pigmentation. When the conductor length is long (3 to 5 mm), the energy reaches deep derma and subcutaneous fat, thereby making it effective for improving cellulite or reducing fat.

In the meantime, the surgical device according to an embodiment of the present disclosure may include an optional cooling system capable of controlling the effective penetration depth of RF energy by adjusting the degree of cooling of the skin surface.

In an embodiment, the surgical device of the present disclosure may include a contact cooling plate, a refrigerant circulation system, and a cooling intensity controller.

The contact cooling plate may adjust the temperature from −5 °C to 15 °C by using a semiconductor cooling element (Peltier).

The refrigerant circulation system uniformly controls the surface temperature of an electrode unit through internal circulating refrigerant.

Under the control of the processor 190, the cooling intensity controller adjusts the cooling intensity stepwise between 0 and 100%.

When the surface cooling intensity is set to be high (80 to 100%), RF energy is induced to penetrate deeper into the tissue by effectively suppressing the temperature rise in the epidermis layer. When the cooling intensity is set to be low (0 to 30%), the epidermis and upper dermal layer may also be treated.

In the meantime, the surgical device according to an embodiment of the present disclosure may include a multi-electrode array system capable of controlling the distribution and penetration depth of RF energy by adjusting the activation pattern and placement of electrodes.

The present disclosure includes a multi-electrode array system capable of controlling the distribution and penetration depth of RF energy by adjusting the activation pattern and placement of electrodes.

In an embodiment, a surgical device of the present disclosure may include a matrix electrode array, an optional electrode activation controller, and a phase control module.

The matrix electrode array may include small electrodes arranged in a 3×3 or 4×4 matrix.

The optional electrode activation controller is a switching circuit that selectively activates only a specific electrode.

The phase control module is a module that adjusts the RF phase between adjacent electrodes in the range of 0 to 180°.

When all the electrodes are activated in the same phase, RF energy is evenly distributed to the epidermis and upper dermal layer. When the phase difference between adjacent electrodes is set to 90 to 180°, a constructive/destructive interference pattern is generated, thereby effectively delivering energy to deep tissues. In particular, when only diagonal electrodes are activated and a 180° phase difference is applied, energy may reach up to the subcutaneous fat layer (the depth of approximately 10 to 15 mm).

In the meantime, the surgical device according to an embodiment of the present disclosure may include a dual-mode system capable of controlling the penetration depth in multiple layers by simultaneously or sequentially irradiating frequencies of 2.45 GHz and 6.78 MHz.

In an embodiment, the surgical device according to an embodiment of the present disclosure may include a dual frequency generator, a frequency mixing module, and a pulse control module.

The dual frequency generator is a module that independently generates RF of 2.45 GHz and 6.78 MHz.

The frequency mixing module adjusts the output ratio between two frequencies in the range of 0 to 100%.

The pulse control module may independently adjust the pulse width and period of each frequency.

The frequency of 2.45 GHz selectively affects the subcutaneous fat layer, while in the frequency of 6.78 MHz, energy is delivered from the superficial skin to the deep derma. An operator may target a specific depth or may treat multiple layers simultaneously by adjusting the ratio between two frequencies (e.g., 2.45 GHz 70%+6.78 MHz 30%).

According to experimental results, when the above-described structural means are complexly used, the penetration depth of RF energy may be precisely adjusted from 1 mm to 15 mm, which allows for a customized procedure for various skin conditions and treatment goals.

Hereinafter, a method for controlling a surgical device according to an embodiment of the present disclosure will be described in detail.

The processor 190 allows the energy supply unit to generate electromagnetic waves of frequencies of 2.40 to 2.50 GHz from the electrode unit during a reference period. In an embodiment, the processor 190 may allow the energy supply unit to generate electromagnetic waves having a frequency of 2.45 GHz.

In the meantime, the processor 190 generates electromagnetic waves from the electrode unit, and the skin cooling time may be variably adjusted depending on a procedure step (e.g., first to fourth sections), a skin temperature, tissue characteristics, or the like. The processor 190 may analyze skin temperature information obtained from the sensing unit 130 in real time to determine the cooling time or control the cooling time according to a preset protocol.

The processor 190 allows the energy supply unit and cooling unit to control the electromagnetic wave generation time and skin cooling time in the electrode unit. Here, the processor 190 may vary the electromagnetic wave generation time and skin cooling time depending on each treatment step.

In an embodiment, the treatment step may be broadly split into four steps. For example, the processor 190 may split the reference period into the first to fourth sections based on a specific point in time within the reference period.

The first section is a treatment start step, where the processor 190 pre-heats the skin by using relatively low power density and a short pulse width. In the first section, while gradually heating the skin, the processor 190 adapts skin tissues to RF energy and minimizes any thermal damage or discomfort that may occur during a procedure.

In an embodiment, the processor 190 generates electromagnetic waves with a pulse width of several milliseconds (ms) to tens of milliseconds (ms), and provides sufficient cooling time between pulses, thereby preventing the skin surface temperature from rising excessively. For example, the processor 190 may have the cooling time of 100 ms after a pulse of 10 ms, or may have the cooling time of 200 ms after a pulse of 50 ms. The specific pulse width and cooling time may be adjusted in consideration of a skin type, a treatment area, patient sensitivity, and the like.

The second section is a main heating step where when the skin adapts to the preheating in the first section, the processor 190 effectively increases the temperature of the tissues deep in the skin by gradually increasing the power density of a pulse, increasing a pulse width, or decreasing the interval (the cooling time) between pulses. In the second section, the processor 190 precisely allows the skin temperature to reach and maintain a target temperature range (e.g., 40 to 45 °C).

In the second section, the processor 190 may control the energy output such that the temperature of the skin does not exceed a reference value.

The processor 190 may allow the cooling unit and the energy supply unit such that the cooling time in the first section is longer than the cooling time in the second section. Moreover, the processor 190 may control the energy supply unit such that the electromagnetic wave generation time in the second section is longer than the electromagnetic wave generation time in the first section. In this way, the present disclosure may allow the skin to adapt to treatment at the beginning of the treatment and may allow the skin's temperature to be safely increased after the skin has adapted.

The processor 190 may continuously monitor the skin's temperature, and may switch to the third section when the skin temperature reaches a target temperature.

The third section is a temperature maintenance step, where under the control of the processor 190, the temperature of the skin is maintained within a certain target temperature range. In the third section, the processor 190 delivers energy to the skin in a continuous wave (CW) method or a pulsed wave method, thereby inducing long-term treatment effects such as collagen regeneration, fat cell destruction, and cellulite improvement in the deep layer of the skin.

In the third section, the processor 190 may control the energy supply unit such that the temperature of the skin is uniformly maintained without cooling the skin. For example, the processor 190 may control the energy supply unit such that lower energy is supplied to the skin compared to the second section.

In an embodiment, the processor 190 may finely adjust the energy output of the energy supply unit by periodically monitoring the temperature of the skin. In this way, the present disclosure may maintain the skin in a stable state during treatment.

The fourth section is a cooling and recovery step. When the preset treatment time in the third section has elapsed or there is a request from a user (operator), the processor 190 switches to the fourth section.

In the fourth section, the processor 190 cools the skin through the cooling unit 140 by stopping the energy supply or gradually reducing the power density or pulse width of the pulse. In this way, the present disclosure safely lowers the temperature of a treatment area, promotes the recovery of a skin tissue, and minimizes post-procedure side effects such as erythema and edema.

In the fourth section, the processor 190 may gradually decrease the pulse time and gradually increase the cooling time such that the temperature of the skin gradually decreases. When the temperature of the skin reaches the target temperature, the processor 190 may terminate the treatment.

In the meantime, the present disclosure may maximize the therapeutic effect by using multiple frequencies.

In an embodiment, the processor 190 may allow the energy supply unit to generate at least one of an electromagnetic wave having frequencies of 2.40 to 2.50 GHz and an electromagnetic wave having frequencies of 6.70 to 6.80 MHz in the electrode unit.

In an embodiment, the processor 190 may allow the energy supply unit to generate electromagnetic waves having the frequencies of 6.70 to 6.80 MHz in the first section, and may allow the energy supply unit to generate electromagnetic waves having the frequencies of 2.40 to 2.50 GHz in the second section. In this way, the present disclosure may secure an adaptation time for the skin by supplying relatively low energy when the skin temperature is low, and may minimize damage to the skin by supplying high energy when the skin has adapted to a procedure.

In an embodiment, the processor 190 may allow the energy supply unit to alternately generate electromagnetic waves having the frequencies of 2.40 to 2.50 GHz and electromagnetic waves having the frequencies of 6.70 to 6.80 MHz in the third section. In this way, while maximizing the treatment effect, the present disclosure may prevent excessive energy from being irradiated to the skin when the skin reaches the target temperature to perform full-scale treatment.

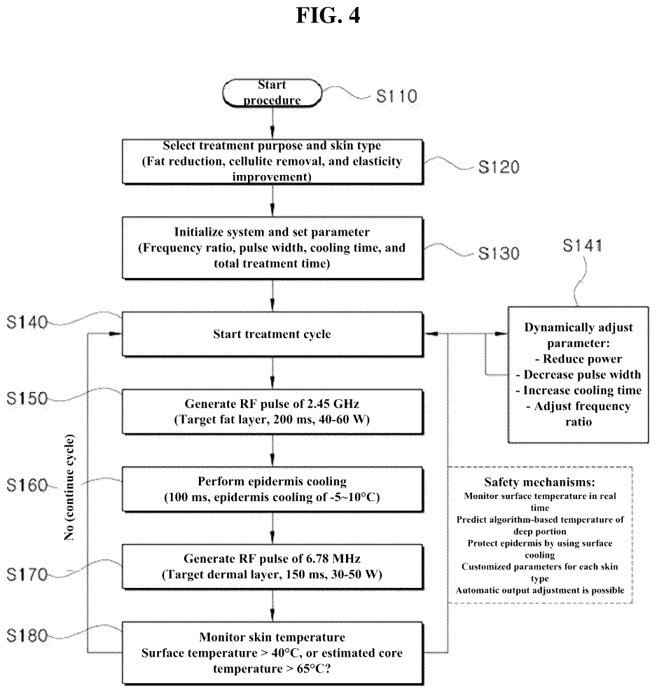

FIG. 4 is a flowchart showing a procedure method utilizing a plurality of frequencies in surface electrode-based RF treatment, according to an embodiment of the present disclosure.

Referring to FIG. 4, when the procedure starts (S110), a step of selecting a treatment purpose and a skin type is performed (S120).

In an embodiment, the treatment purpose may include options for fat reduction, cellulite removal, and elasticity improvement.

Afterwards, a step in which the processor 190 initializes a system and sets a parameter is performed (130).

The processor 190 may set a frequency ratio, a pulse width, a cooling time, and a total treatment time, which are suitable for the treatment purpose and skin type.

Afterwards, a treatment cycle starts (S140).

When the treatment cycle starts, the processor 190 may dynamically adjust a parameter by monitoring a skin condition (S141). In detail, the processor may perform power reduction, a decrease in a pulse width, an increase in a cooling time, the adjustment of a frequency ratio, or the like.

When the treatment cycle starts, the processor 190 generates a RF pulse of 2.45 GHz (S150). In an embodiment, the frequency may target a fat layer and may supply energy of 40 to 60 W having a pulse of 200 ms.

Afterwards, the processor 190 performs epidermis cooling (S160). In an embodiment, the epidermis cooling may cool the epidermis to a temperature of −5 to 10 °C for 100 ms.

Next, the processor 190 generates a RF pulse of 6.78 MHz (S170). In an embodiment, the frequency may target a dermal layer and may supply energy of 30 to 50 W having a pulse of 150 ms.

While the procedure is in progress, the processor 190 monitors a skin temperature (S180). In an embodiment, when a surface temperature exceeds 40 °C or the estimated core temperature exceeds 65 °C, the processor 190 performs epidermis cooling to prevent damage to a skin.

In the meantime, the present disclosure may provide a multi-frequency mode in which RF energy of different frequencies is alternately irradiated during the first to fourth sections. For example, in the first section, a skin warming operation may be performed by using a relatively low frequency (e.g., 6.78 MHz); in the second and third sections, an operation of achieving main therapeutic effects may be performed by using 2.45 GHz; and, in the fourth section, a skin calming operation may be performed again by using a low frequency. This multi-frequency mode may be further subdivided according to skin type or treatment purpose, and power density, a pulse width, a cooling time, or the like for each frequency may be adjusted independently.

In the meantime, the present disclosure may implement various treatment modes utilizing electromagnetic waves of a frequency of 2.40 to 2.50 GHz.

In an embodiment, the process may implement a treatment mode for increasing skin elasticity and improving wrinkles. In detail, the processor 190 may continuously irradiate energy with medium or high output such that the energy may reach middle and deep layers of the skin to promote collagen regeneration. The processor 190 may monitor the skin temperature in real time to prevent the skin from overheating and may continuously irradiate energy to promote natural healing of the skin.

Treatment for skin elasticity and wrinkle improvement using a surgical device according to an embodiment of the present disclosure is performed through the following mechanisms The RF energy of 2.45 GHz is absorbed by a skin dermal layer and generates heat, and the heat induces shrinkage and degeneration of collagen fibers. During this process, old or damaged collagen is removed and new collagen production is promoted. Moreover, thermal stimulation activates fibroblasts, which increases the synthesis of various extracellular matrix (ECM) components, including elastin and hyaluronic acid as well as collagen. As a result, the skin elasticity is increased, wrinkles are improved, and skin texture is smoothed.

In an embodiment, the processor 190 may implement a treatment mode for improving cellulite and fat accumulation. In detail, the processor 190 may intensively heat fat cells by periodically irradiating short pulses of strong energy, and may promote fat decomposition. The processor 190 may shortly irradiate a pulse of high energy to deliver sufficient heat to the fat layer and may cool the skin surface during a sufficient time to prevent skin damage.

In an embodiment, a cellulite and fat reducing mode operates in a method of selectively destroying fat cells by focusing heat on a subcutaneous fat layer by using high-frequency RF energy of 2.45 GHz. 2.45 GHz has a high absorption rate for water molecules, which effectively generates heat in adipose tissues, thereby inducing apoptosis or necrosis of fat cells. The destroyed fat cells are naturally eliminated through the body's metabolic processes, which reduces the thickness of the fat layer in the procedure area and improves cellulite. Moreover, the RF energy also helps to relax fibrous septae around fatty tissues, thereby reducing the roughness of the skin surface.

In an embodiment, the processor 190 may implement a treatment mode for reducing chronic pain. In detail, the processor 190 may continuously irradiate low energy capable of reaching deep tissues. In this way, the present disclosure may relieve muscle tension and increase blood flow. The processor 190 may continuously supply low energy such that energy is capable of being delivered to deep muscle areas, and may prevent the skin temperature from exceeding a preset temperature.

In an embodiment, the chronic pain relief mode operates in a method of delivering heat to deep tissues (muscles, ligaments, joints, etc.) by using RF energy of 2.45 GHz, and thus relieving pain. The RF energy has the effect of dilating blood vessels to increase a blood flow, relieving muscle spasms, and decreasing the sensitivity of nerve endings. Furthermore, heat stimulation may contribute to relieving chronic pain by promoting the release of pain-inhibiting substances (e.g., endorphins) and modulating inflammatory responses.

In an embodiment, the processor 190 may implement a treatment mode for permanently removing hair. In detail, the processor 190 may focus high-frequency energy of 2.45 GHz on the skin depth (approximately 1-3 mm) corresponding to a location of a hair follicle to precisely increase the temperature around the hair follicle to 65 to 72 °C. This temperature range is optimal for inhibiting hair regeneration by causing thermal damage to hair follicle stem cells and vascular structures. In addition, while maximizing epidermis cooling to protect the epidermis and reduce pain, the processor 190 may selectively target hair follicles including melanin pigment through precise energy control. In an embodiment, the hair removal mode operates in a method of effectively removing hair in the anagen phase of the hair follicle by using the characteristics of RF energy of 2.45 GHz. The RF energy of 2.45 GHz is selectively absorbed by water and melanin in the hair follicle, thereby causing irreversible damage to the hair follicle stem cells. Unlike conventional laser hair removal, RF-based hair removal is effective regardless of skin and hair color, and is especially effective on fine or light-colored hair. In addition, according to an embodiment of the present disclosure, the structure of a variable impedance matching circuit and a coaxial antenna may focus energy on the exact depth of the hair follicle, thereby achieving a higher permanent hair removal rate (the average of 85 to 95%) compared to conventional RF hair removal technologies.

In an embodiment, the processor 190 may implement a treatment mode for managing a skin disease. In detail, the present disclosure may reduce inflammation of the skin and may stimulate a healing process by continuously irradiating low energy. The processor 190 may reduce skin inflammation by continuously irradiating low energy to the inflamed skin area.

In an embodiment, the skin disease management mode operates in a method of alleviating symptoms of various skin diseases (acne, atopic dermatitis, psoriasis, etc.) by adjusting inflammatory responses by using the RF energy of 2.45 GHz, promoting skin regeneration, and providing antibacterial effects. The RF energy suppresses the production of inflammatory mediators, regulates the activity of immune cells, and promotes the repair of damaged tissues. Moreover, 2.45 GHz may have antibacterial effects against some bacteria (e.g., acne bacteria), which may help treat inflammatory skin conditions.

In an embodiment, the processor 190 may implement a treatment mode for skin lifting. The present disclosure may continuously supply energy having a middle level to stimulate collagen production and to restore the elasticity of the skin. Under the control of the processor 190, heat may be delivered to deep skin areas while heat distribution is uniformly maintained.

In an embodiment, a skin lifting mode operates in a method of contracting collagen fibers in a dermal layer by using the RF energy of 2.45 GHz, stimulating new collagen production to increase skin elasticity, improving wrinkles, and sharpening contours of a face. The RF energy heats collagen fibers to cause immediate contraction, and in the long term, stimulates fibroblasts to increase the synthesis of new collagen and elastin fibers. These effects help tighten the skin, improve sagging skin, and smooth out fine and deep wrinkles.

As described above, a surgical device according to the above problem-solving means of the present disclosure irradiates a high frequency of 2.45 GHz. The high frequency of 2.45 GHz has the high absorption rate of water molecules, and thus energy absorption occurs intensively in the fat layer. In this way, the present disclosure may improve the fat reduction and cellulite treatment effects by focusing energy on skin tissue where fat is concentrated.

Moreover, RF of 2.45 GHz has a shorter wavelength than conventional RF, which allows the handpiece size of the surgical device to be made smaller and adaptable to small treatment areas.

Hereinafter, the effects identified after a procedure is performed by using a surgical device according to an embodiment of the present disclosure will be described.

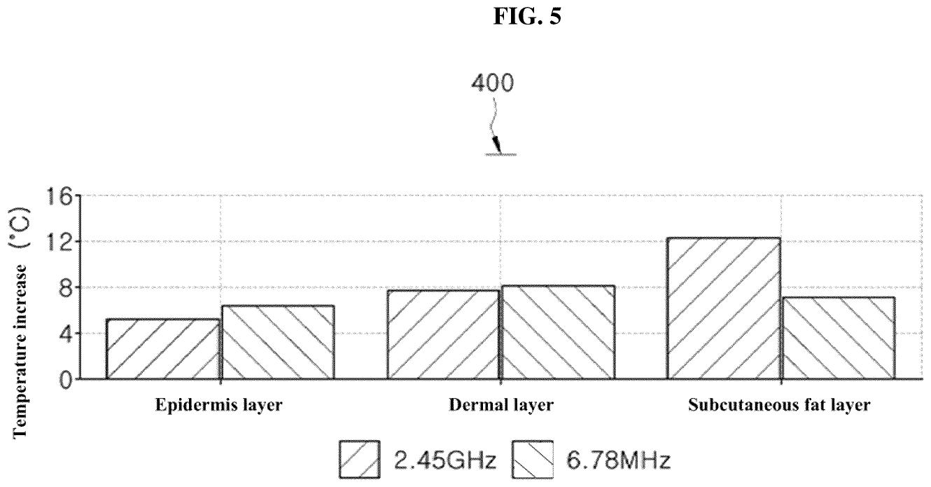

FIGS. 5 to 7 are graphs showing treatment effects during surface electrode-based RF treatment, according to an embodiment of the present disclosure.

First of all, to identify the procedure effect of a high frequency of 2.45 GHz, RF of 2.45 GHz and RF of 6.78 MHz are irradiated to each skin layer for 60 seconds, and then a temperature change is observed. In detail, the experiment is conducted on a multilayer phantom model similar to human skin, and the permittivity of each skin layer is epidermis (38.0), derma (43.0), and subcutaneous fat (10.8). Temperature sensing is performed by using a thermal imaging camera and an optical fiber temperature sensor.

Referring to FIG. 5, the RF of 2.45 GHz showed the highest temperature increase (12.3 °C) in a subcutaneous fat layer, and it is identified that the RF of 2.45 GHz has superior fat selectivity compared to the RF of 6.78 MHz.

In particular, it is identified that the subcutaneous fat was reduced by 14.8% after 4 procedures. In this way, it is identified that a surgical device according to an embodiment of the present disclosure is effective in reducing fat. Cellulite severity is evaluated by using standardized Cellulite Severity Scale (CSS) (0 to 15 points), and the CSS decreased from a mean score of 9.7±1.2 before the procedure to 4.7±0.9 after 4 procedures (p<0.001). In particular, it is noted that all subjects (25/25, 100%) show at least 3-point or more CSS score reduction, and 18 subjects (72%) show a significant improvement of 5 points or more. The thickness of the subcutaneous fat is measured by using high-resolution ultrasound (10 MHz) based on a mean value measured three times for each procedure area. The average subcutaneous fat thickness before the procedure is 28.4±4.2 mm and then decreases to 24.2±3.6 mm after 4 procedures. This indicates a statistically significant reduction rate of 14.8% (p<0.001). Besides, subcutaneous fat density (echogenicity) analysis results indicates that the size of a fat cell decreases by an average of 32.6%. The result of using a cutometer to measure skin elasticity indicates that elastic recovery (R2 parameter) is improved by 26.7±3.8% compared to before the procedure, which is a result that exceeds the improvement rate (15 to 20%) generally observed after the conventional RF procedure of 6.78 MHz.

In the meantime, after a procedure including the first to fourth sections described above is performed multiple times, the change in cellulite severity and the reduction rate of subcutaneous fat are observed.

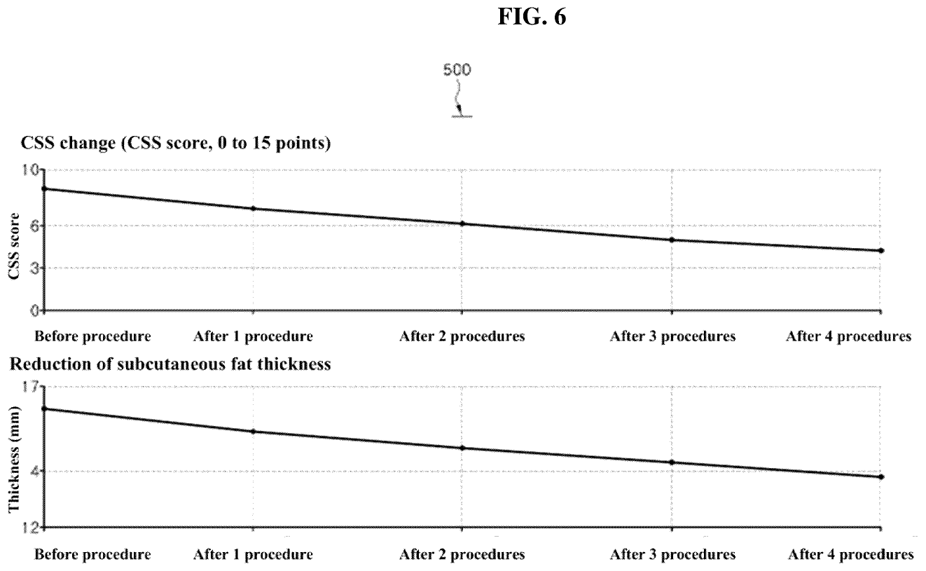

Referring to FIG. 6, it is identified that the CSS score decreases as the procedure is repeated. In particular, it is identified that the cellulite severity is reduced by 51.2% after four procedures. In this way, it is identified that a surgical device according to an embodiment of the present disclosure is effective in reducing cellulite. Moreover, it is identified that the subcutaneous fat thickness decreased as the procedure is repeated.

In particular, it is identified that the subcutaneous fat was reduced by 14.8% after 4 procedures. In this way, it is identified that a surgical device according to an embodiment of the present disclosure is effective in reducing fat. Cellulite severity is evaluated by using standardized Cellulite Severity Scale (CSS) (0 to 15 points), and the CSS decreased from a mean score of 9.7±1.2 before the procedure to 4.7±0.9 after 4 procedures (p<0.001). In particular, it is noted that all subjects (25/25, 100%) show at least 3-point or more CSS score reduction, and 18 subjects (72%) show a significant improvement of 5 points or more. The thickness of the subcutaneous fat is measured by using high-resolution ultrasound (10 MHz) based on a mean value measured three times for each procedure area. The average subcutaneous fat thickness before the procedure is 28.4±4.2 mm and then decreases to 24.2±3.6 mm after 4 procedures. This indicates a statistically significant reduction rate of 14.8% (p<0.001). Besides, subcutaneous fat density (echogenicity) analysis results indicates that the size of a fat cell decreases by an average of 32.6%. The result of using a cutometer to measure skin elasticity indicates that elastic recovery (R2 parameter) is improved by 26.7±3.8% compared to before the procedure, which is a result that exceeds the improvement rate (15 to 20%) generally observed after the conventional RF procedure of 6.78 MHz.

Meanwhile, the treatment effects are observed after 25 subjects aged 35 to 65 years underwent facial procedures three times at two-week intervals.

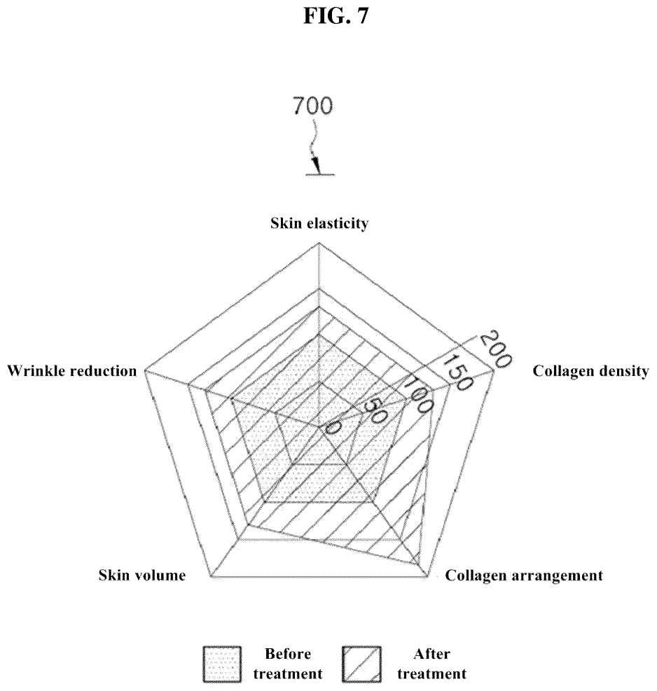

Referring to FIG. 7, after 3 procedures, collagen density increases by an average of 32.7% and skin elasticity is improved by 26.3%.

In this way, it is identified that in the case of RF of 2.45 GHz, the skin elasticity is improved by stimulating collagen production through thermal shock protein activation and inducing existing collagen contraction. It is identified that through skin histological analysis, collagen density increases by 32.7% from 14.8±2.6% before the procedure to 47.5±4.2% after the procedure (p<0.001). The arrangement of collagen fibers also improved from an irregular and fragmented form before the procedure to a dense and aligned form after the procedure. It is showed that through electron microscopic observations, the collagen fiber diameter increases by 50% from an average of 52±8 nm before the procedure to 78±6 nm after the procedure. This is a factor that directly contributes to the improvement of skin strength and elasticity. In the skin elasticity test, parameter R2 (pure elasticity) using a cutometer is improved by 26.3% from 0.64±0.07 before the procedure to 0.81±0.05 after the procedure, and parameter R7 (biological elasticity) also increases by 35.8% from 0.53±0.08 to 0.72±0.06 (p<0.001). In the subject satisfaction questionnaire (Visual Analogue Scale, 0 to 10 points) after the procedure, the average satisfaction score indicates 8.4±0.7, and 23 subjects (92%) expresses high satisfaction of 7 points or more. The pain index is measured at an average of 3.2±1.1, which is significantly lower than that of the conventional RF procedure (average of 5.7±1.4).

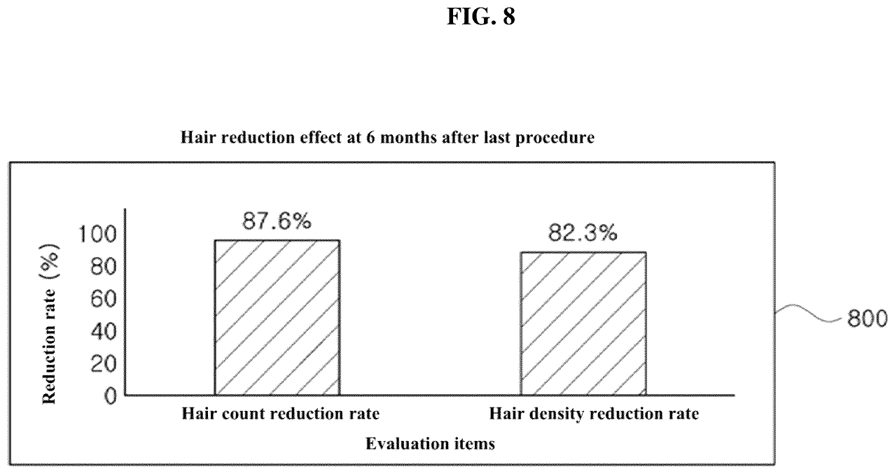

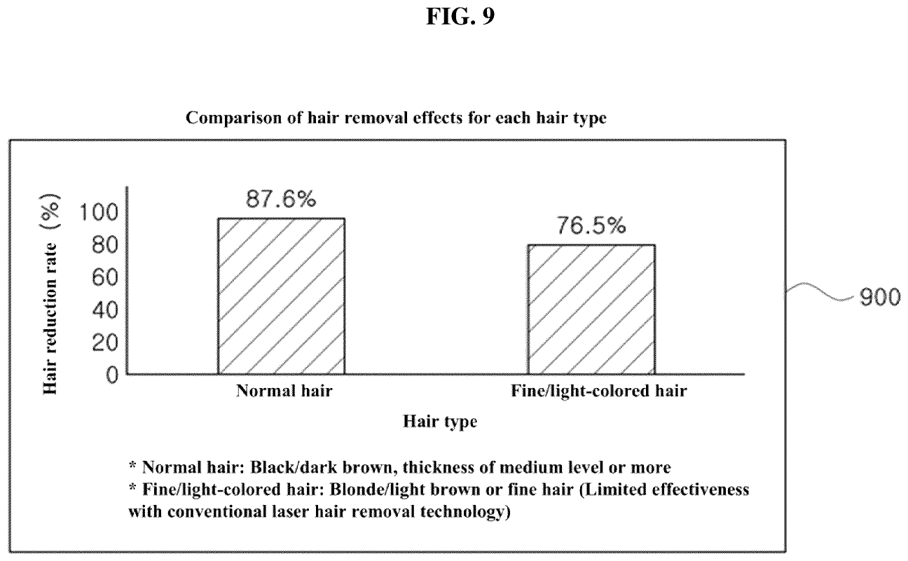

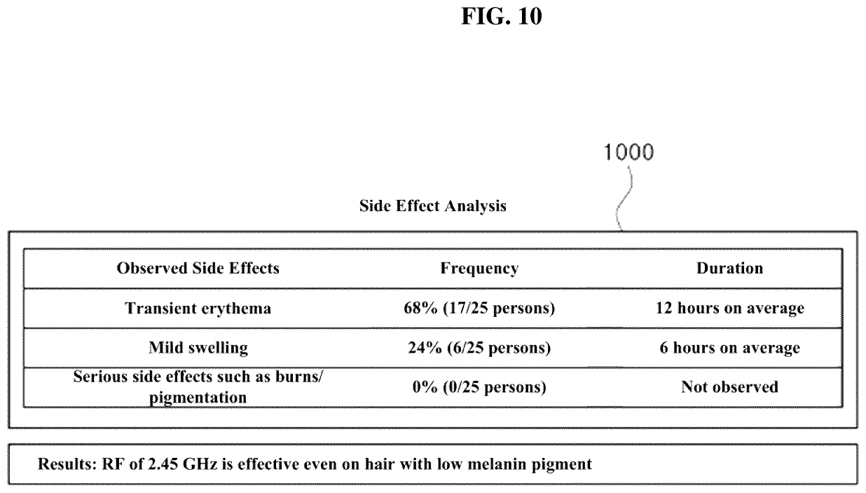

Furthermore, the hair removal effect was evaluated on 25 subjects (skin types II-V, various hair colors and thicknesses). The subjects underwent a total of four procedures at four-week intervals, and the hair loss rate was evaluated at six months after the last procedure. As a result, at 6 months after the last procedure, the average hair count reduction rate indicates 87.6%, and hair density decreases by 82.3% compared to before the procedure (see FIG. 8). In particular, a significant hair removal effect (76.5% hair reduction rate) is observed even on fine hair or light-colored hair, which was limited in effectiveness with a conventional laser hair removal technology (see FIG. 9). This suggests that RF of 2.45 GHz is capable of being effective even on hair with low melanin pigmentation. In terms of side effects, transient erythema (average duration of 12 hours) is the most common, and no serious side effects such as burns or pigmentation are observed (see FIG. 10).

As a result, it is identified that the RF of 2.45 GHz is effective even on hair with low melanin pigment.

In the meantime, to verify the effectiveness of the penetration depth control mechanism according to an embodiment of the present disclosure, experiments using various tissue phantoms and ex-vivo human skin samples are conducted. The drawings attached to this specification illustrate the results of the experiments.

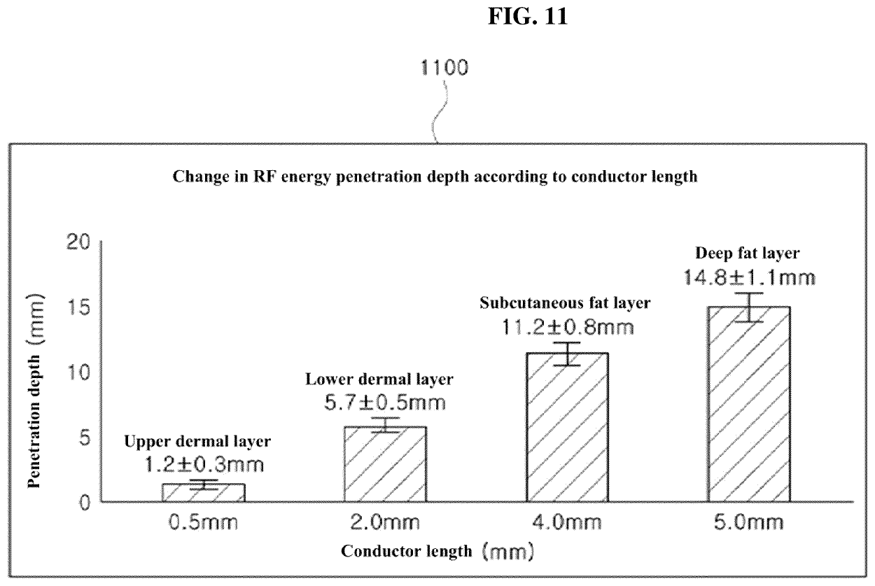

FIG. 11 is a conceptual diagram showing a change in penetration depth according to conductor length adjustment.

Referring to FIG. 11, it may be seen that there is a clear correlation between a conductor length and a penetration depth. When the conductor length is 0.5 mm, the penetration depth is limited to an upper dermal layer of 1.2±0.3 mm. On the other hand, as the conductor length increases, the penetration depth also increased. The penetration depth of 14.8±1.1 mm is achieved at a conductor length of 5.0 mm. This means that RF energy effectively reaches a deep fat layer.

The present experiments demonstrate that energy transfer is capable of being optimized depending on the depth of a target tissue simply by precisely adjusting the conductor length. In particular, it shows that the error range of the penetration depth for each conductor length is relatively small, and thus precise control is possible.

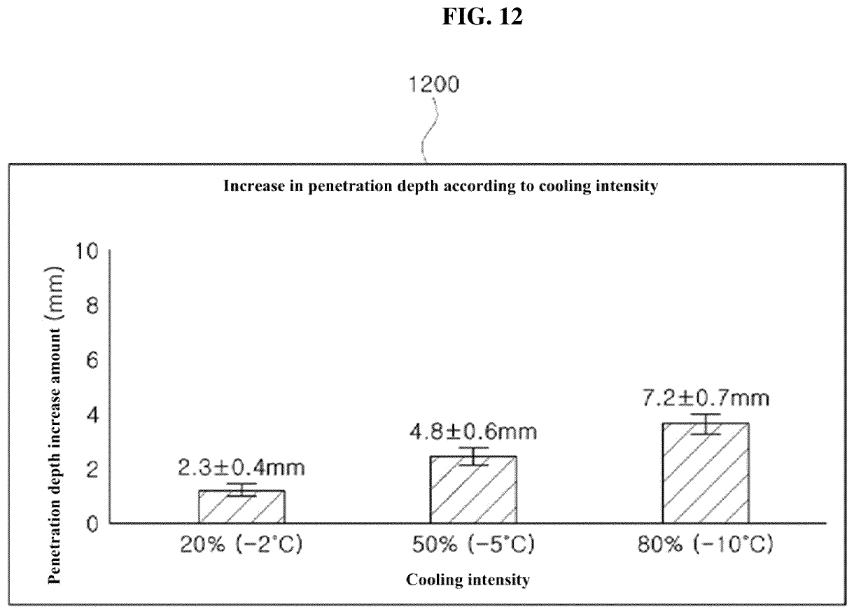

FIG. 12 is a graph showing a change in penetration depth according to cooling intensity.

Referring to FIG. 12, when an epidermis cooling technology is applied, an effective penetration depth tends to increase as the cooling intensity increases. When the cooling intensity is 20% (−2 °C) , the penetration depth increased by 2.3±0.4 mm. When the cooling intensity increased to 80% (−10 °C) , the penetration depth increased to 7.2±0.7 mm.

These results scientifically demonstrate that the epidermis cooling mechanism allows RF energy to be delivered to deeper layers while minimizing thermal damage to superficial tissues. The effective penetration depth may be further controlled by precisely adjusting the cooling intensity, thereby allowing for more precise depth adjustment when it is used together with conductor length adjustment.

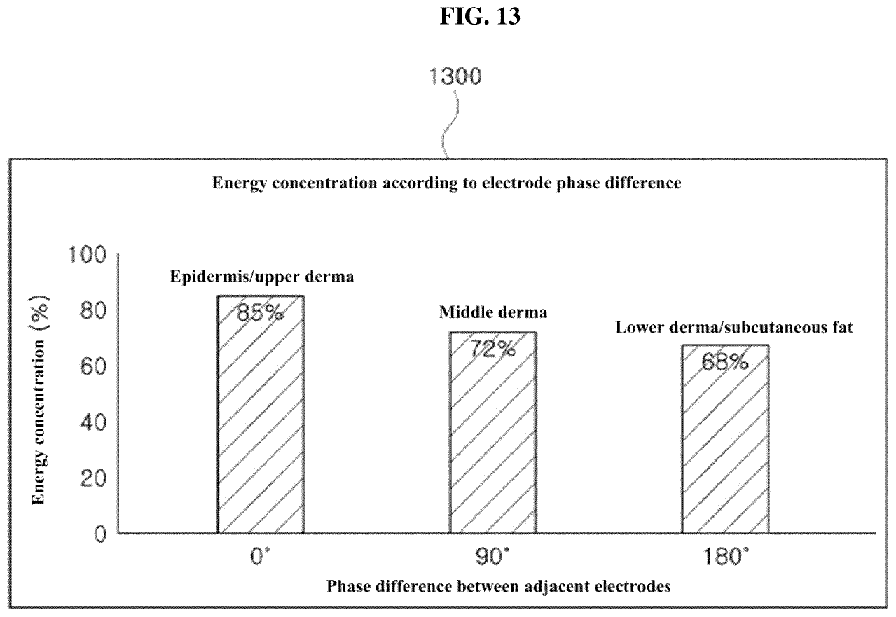

FIG. 13 is a graph showing energy concentration according to an electrode phase difference.

Referring to FIG. 13, it is shown that a skin layer where RF energy is concentrated may be selectively targeted by adjusting a phase difference. When the phase difference between adjacent electrodes is 0°, 85% of the energy is concentrated in an epidermis/upper derma; when the phase difference between adjacent electrodes is 90°,72% of the energy is concentrated in a middle derma; and when the phase difference between adjacent electrodes is 180°,68% of the energy is concentrated in a lower derma/subcutaneous fat.

This phase difference control mechanism means that energy may be selectively focused on target tissue layers even when the same RF energy is used, enabling optimized energy transfer for various skin procedure purposes.

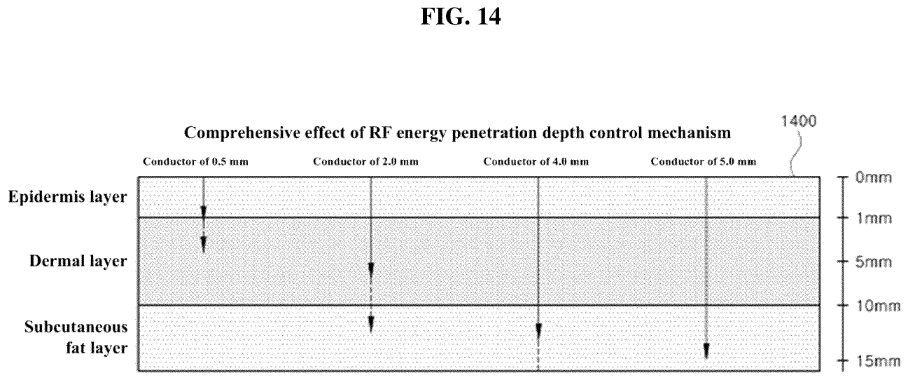

FIG. 14 is a graph showing a comprehensive effect of an RF energy penetration depth control mechanism.

Referring to FIG. 14, a basic penetration depth (solid arrow) along a conductor length on a skin cross-section and an extended penetration depth (dotted arrow) in the case where cooling intensity and phase difference control are additionally applied are shown.

The penetration depth may be adjusted from 1 mm to a maximum of 15 mm only by controlling the conductor length, and it is visually showed that more precise energy transfer and depth control are possible when cooling intensity and phase difference control are complexly applied.

As described above, a surgical device according to an embodiment of the present disclosure has more precise penetration depth control performance than conventional RF devices. In particular, the combination of a coaxial antenna structure and a variable impedance matching circuit achieves the energy transfer efficiency of 90% or more at a target depth while energy loss is minimized, which significantly exceeds the transfer efficiency (approximately 60 to 70%) of conventional technologies.

In detail, a composite control mechanism according to an embodiment of the present disclosure of provides the following technical advantages.
1. Precise penetration depth control: Precisely controlling over a wide range of penetration depths from 1 mm to 15 mm
2. Selective energy concentration: Selectively concentrating energy on specific skin layers by adjusting a phase difference
3. Surface protection: Improving protection functions of epidermis and upper derma through adjusting intensity control
4. High energy transfer efficiency: Maximizing procedure effects with energy transfer efficiency of 90% or more
5. Customized procedures available: Optimizing parameters to be suitable for various skin conditions and treatment purposes.

These technical features provide an innovative approach that simultaneously improves the effectiveness and safety of RF energy-based skin procedures.

Meanwhile, the disclosed embodiments may be implemented in a form of a recording medium storing instructions executable by a computer. The instructions may be stored in a form of program codes, and, when executed by the processor 190, generate a program module to perform operations of the disclosed embodiments. The recording medium may be implemented as a computer-readable recording medium.

The computer-readable recording medium may include all kinds of recording media in which instructions capable of being decoded by a computer are stored. For example, there may be read only memory (ROM), random access memory (RAM), magnetic tape, magnetic disk, flash memory, optical data storage device, and the like.

Disclosed embodiments are described above with reference to the accompanying drawings. One ordinary skilled in the art to which the present disclosure belongs will understand that the present disclosure may be practiced in forms other than the disclosed embodiments without altering the technical ideas or essential features of the present disclosure. The disclosed embodiments are examples and should not be construed as limited thereto.

According to the above problem-solving means of the present disclosure, a surgical device irradiates a high frequency of 2.45 GHz. The high frequency of 2.45 GHz has the high absorption rate of water molecules, and thus energy absorption occurs intensively in the fat layer. In this way, the present disclosure may improve the fat reduction and cellulite treatment effects by focusing energy on skin tissue where fat are concentrated.

The surgical device of the present disclosure may provide an effective hair removal effect by selectively heating pigment cells inside hair follicles by irradiating high frequency of 2.45 GHz. Moreover, hair follicles may be precisely targeted through precise penetration depth control while damage to surrounding tissue is minimized. Besides, a cooling system of the present disclosure may effectively protect the epidermis during a hair removal procedure, thereby significantly reducing pain and side effects.

RF of 2.45 GHz has a shorter wavelength than conventional RF, which allows the handpiece size of the surgical device to be made smaller and adapted to small treatment areas.

Effects of the present disclosure are not limited to the effects mentioned above, and other effects not mentioned will be apparent by those skilled in the art from the following description.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. A surgical device capable of delivering high-frequency energy during surface electrode-based radio frequency (RF) treatment, the surgical device comprising:
an electrode unit configured to touch a surface of a skin;
an energy supply unit configured to supply energy to the electrode unit during a reference period;
a cooling unit; and
a processor configured to control the energy supply unit and the cooling unit, wherein the electrode unit includes a coaxial antenna structure composed of a central conductor, a dielectric layer, and an outer conductor,
wherein the energy supply unit includes an RF energy generation system including an oscillator and amplifier, which are designed to operate stably at 2.40 to 2.50 GHz, and a variable impedance matching circuit configured to automatically adjust antenna impedance,
wherein the cooling unit includes at least one of contact cooling using a semiconductor cooling element and non-contact cooling using refrigerant injection, and
wherein the processor is configured to:
control the energy supply unit such that an electromagnetic wave of a frequency of 2.40 to 2.50 GHz is generated by the electrode unit.

2. The surgical device of claim 1, wherein the processor is configured to:
allow the cooling unit to cool the skin during a predetermined time after an electromagnetic wave is generated by the electrode unit.

3. The surgical device of claim 1, wherein the processor is configured to:
split the reference period into a first section and a second section based on a specific point in time within the reference period; and
control the cooling unit and the energy supply unit such that a cooling time in the first section is different from a cooling time in the second section.

4. The surgical device of claim 3, wherein the processor is configured to:
control the cooling unit and the energy supply unit such that the cooling time in the first section is longer than the cooling time in the second section.

5. The surgical device of claim 3, wherein the processor is configured to:
control the energy supply unit such that an electromagnetic wave generation time in the second section is longer than an electromagnetic wave generation time in the first section.

6. The surgical device of claim 3, further comprising:
a temperature sensor configured to measure a temperature of a procedure area of the skin,
wherein the processor is configured to:
switch to a third section when a temperature of the skin in the second section reaches a target temperature; and
supply low energy compared to the second section such that a temperature of the skin is uniformly kept without a cooling time in the third section.

7. The surgical device of claim 6, wherein the processor is configured to:
switch to a fourth section when a preset time elapses after switching to the third section, or there is a user request; and
control the energy supply unit and the cooling unit such that the temperature of the skin gradually decreases in the fourth section.

8. The surgical device of claim 1, wherein the processor is configured to:
control the energy supply unit such that at least one of the electromagnetic wave of the frequency of 2.40 to 2.50 GHz and an electromagnetic wave of a frequency of 6.70 to 6.80 MHz is generated by the electrode unit.

9. The surgical device of claim 3, wherein the processor is configured to:
control the energy supply unit such that the electromagnetic wave of the frequency of 6.70 to 6.80 MHz is generated in the first section; and
control the energy supply unit such that the electromagnetic wave of the frequency of 2.40 to 2.50 GHz is generated in the second section.

10. The surgical device of claim 6, wherein the processor is configured to:
control the energy supply unit such that the electromagnetic wave of the frequency of 2.40 to 2.50 GHz and the electromagnetic wave of the frequency of 6.70 to 6.80 MHz are alternately generated in the third section.

11. The surgical device of claim 1, wherein the cooling unit is configured to:
cool the skin by spraying a refrigerant containing R-134a (1,1,1,2-Tetrafluoroethane) or HFO-1234ze (trans-1,3,3,3-Tetrafluoropropene) onto the skin.

12. The surgical device of claim 1, wherein the cooling unit is configured to:
cool the skin by combining a contact cooling method using a Peltier element, and a cooling method of spraying a refrigerant containing R-134a or HFO-1234ze onto the skin.

13. The surgical device of claim 1, wherein the processor is configured to:
perform a treatment mode for hair removal, and
wherein in the treatment mode, the processor is configured to:
control the electromagnetic wave of the frequency of 2.40 to 2.50 GHz to increase a temperature around a hair follicle to a range of 55 to 72 °C; and
maintain a skin surface temperature in a range of 10 to 20 °C through the cooling unit to protect epidermis.
